(19) United States
(12) Patent Application Publication
(10) Pub. No.: US 2006/0020200 A1
(43) Pub. Date: Jan. 26, 2006

(54) Artifact-free CT angiogram

(76) Inventors: Joshua Eric Medow, Madison, WI (US); Charles Anthony Mistretta, Madison, WI (US); Ranjini P. Tolakanahalli, Madison, WI (US); Jiang Hsieh, Brookfield, WI (US)

Correspondence Address: QUARLES & BRADY LLP, 411 E. WISCONSIN AVENUE, SUITE 2040, MILWAUKEE, WI, 53202-4497, US

(21) Appl. No.: 10/887,489

(22) Filed: Jul. 8, 2004

(51) Int. Cl. A61B 5/05

(52) U.S. Cl. 600/425

(57) ABSTRACT

A helical scan is performed with a fan beam or cone beam CT system to acquire a first set of sinogram data sets. The subject is injected with a contrast agent and the identical helical scan is performed to acquire a second set of sinogram data sets. Corresponding projection views are subtracted in the two data sets and a number of different images are reconstructed from the difference data set. One image is a tomographic image produced using a filtered backprojection method and a second image is a topograph produced by selecting and displaying projection views acquired at the same view angle in successive sinogram data sets.

### STATEMENT REGARDING FEDERALLY SPONSORED RESEARCH

This invention was made with government support under Grant No. NIH EB001683 awarded by the National Institute of Health. The United States Government has certain rights in this invention.

### BACKGROUND OF THE INVENTION

The field of the invention is angiography, and particularly the production of angiograms using an x-ray CT system. Medical diagnostic imaging, is generally provided by CT, ultrasound, and MR systems, as well as those using positron emission tomography (PET), and other techniques. One particularly desirable use for such systems is the imaging of blood vessels in a patient, i.e. vascular imaging. Vascular imaging methods include two-dimensional (2D) techniques, as well as reconstruction of three-dimensional (3D) images from 2D image data acquired from such diagnostic imaging systems. In CT medical diagnosis, for example, 3D reconstruction of computed tomograms is particularly useful for visualizing blood vessels.

Conventional digital subtraction angiography (DSA) is considered the most accurate technique for medical diagnosis of vascular structures and remains the standard against which other methods are compared. However, conventional angiography is an invasive technique in which arterial catheterization and injection of a contrast agent presents a certain amount of risk. Accurate evaluation of the vascular system with noninvasive techniques remains an important goal. Thus, duplex ultrasound is often used for evaluation of blood flow in carotid arteries. Magnetic resonance angiography is also used for detailed evaluation of the vascular system. However, both of these techniques have limitations and alternative noninvasive approaches continue to be investigated.

Spiral computed tomography (CT) is a relatively new approach to CT that allows continuous data collection while a subject is advanced through the CT gantry. This provides an uninterrupted volume of x-ray attenuation data. From this data, multiple contiguous or overlapping slices of arbitrary thickness can be reconstructed. Spiral CT permits acquisition of a large volume of data in seconds. With spiral CT angiography (CTA), vascular structures can be selectively visualized by choosing an appropriate delay after IV injection of a contrast material. This gives excellent visualization of vessel lumina, stenoses, and lesions. The acquired data can then be displayed using 3D visualization techniques (e.g., volume-rendering, maximum intensity projection (MIP), and shaded surface display) to give an image of the vasculature. In contrast to conventional angiography, CTA is three-dimensional, thus giving the viewer more freedom to see the vasculature from different viewpoints.

There are a number of disadvantages of CTA as compared to DSA. First, when metal objects such as aneurysm clips or coils are in the field of view troublesome metal artifacts are produced in the image by the tomographic reconstruction process. Also, DSA (1024×1024 pixels) has four times the resolution of CT systems (512×512 pixels) allowing tiny abnormalities to be obscured when using CTA.

### SUMMARY OF THE INVENTION

The present invention is a method for producing an angiogram with an x-ray CT system which is not obscured by metal artifacts and which can rival the resolution of a DSA image. More particularly: a data set is acquired with a CT system which includes a plurality of slices disposed along an axis in which each slice data subset includes a plurality of projections acquired at a corresponding plurality of gantry angles; a topographic plane data set is formed at a selected gantry angle by selecting from each slice data subset the projection corresponding to the selected gantry angle; and a 2D topographic image is produced by displaying the selected projections in the topographic plane data set at their corresponding slice locations along the axis. An angiogram is produced by acquiring one data set before contrast injection, acquiring the same data set after contrast injection and then subtracting the corresponding projections in each data set.

Another aspect of the present invention is to produce a CT image in which metal artifacts are significantly suppressed. More particularly: a first data set is acquired before injection of a contrast agent which includes a series of projections acquired at a succession of gantry angles and a succession of locations along an axis; a second data set is acquired after contrast injection which includes a series of projections acquired at the same succession of gantry angles and succession of locations along the axis as the first data set; a difference data set is produced by subtracting projections in the first data set from the corresponding projections in the second data set; and a tomographic image is produced by tomographically reconstructing the image from the difference data set. Signals caused by metal objects in the field of view are suppressed by subtracting projections from the two acquired data sets before they have an opportunity to affect the tomographic image reconstruction process.

### BRIEF DESCRIPTION OF THE DRAWINGS

FIG. 1 is a pictorial view of an x-ray CT system which employs the present invention;

FIG. 2 is a block diagram of the CT system of FIG. 1;

FIG. 3 is a perspective view of a third generation gantry assembly used in the CT system of FIG. 1;

FIG. 4 is a perspective view of a fourth generation gantry assembly used in the CT system of FIG. 1;

FIG. 5 is a schematic view of a fan beam projection view acquired with the gantry assembly of FIG. 3 or FIG. 4;

FIG. 6 is a schematic view of a sinogram data set formed by storing projection views acquired by the CT system of FIG. 1;

FIG. 7 is a pictorial representation of a helical scan performed with a cone beam x-ray source and a two-dimensional detector array;

FIG. 8 is a pictorial representation of the helical path;

FIG. 9 is a schematic representation of a helical scan path showing the projection views selected to form a topographic plane data set according to one embodiment of the present invention;

FIG. 10 is a schematic representation of sinogram data sets acquired during the helical scan of FIG. 9 showing the projection views selected for a topograph;

FIG. 11 is a pictorial representation of the topographic plane data set and the resulting reconstructed topograph image; and

FIG. 12 is a flow chart illustrating a preferred method for practicing the present invention.

### DETAILED DESCRIPTION OF THE PREFERRED EMBODIMENT

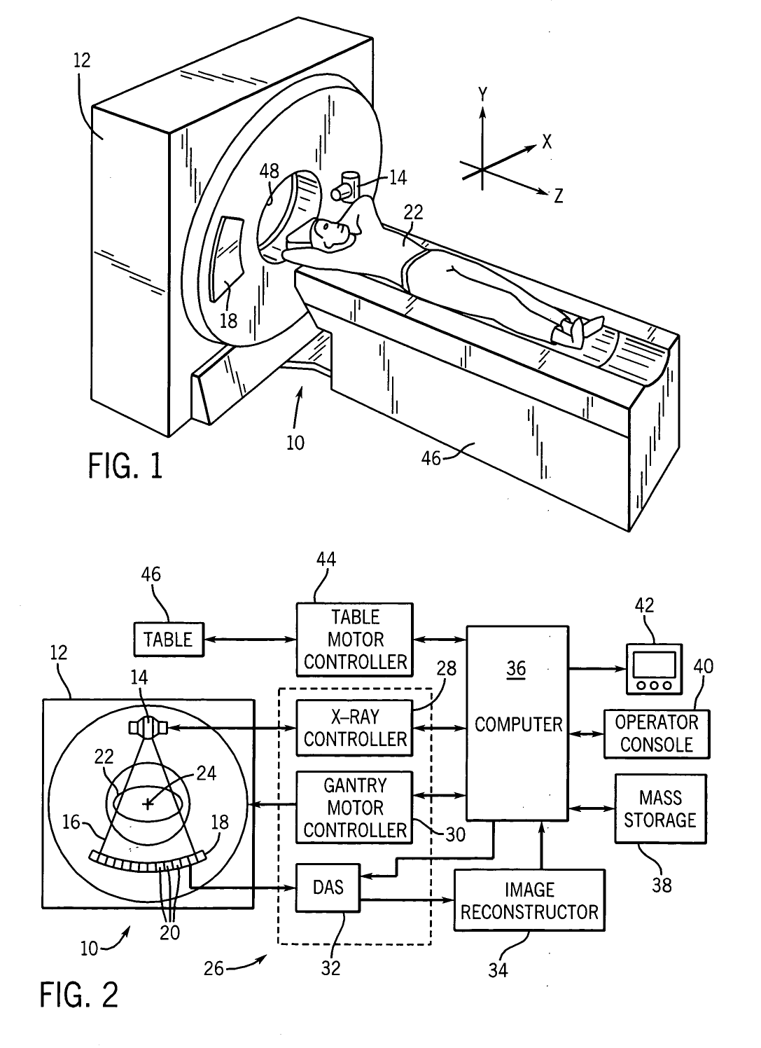

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector array 18 on the opposite side of gantry 12. Detector array 18 is formed by detector elements 20 which together sense the projected x-rays that pass through an object 22, for example a medical patient. Detector array 18 may be fabricated in a single slice or multi-slice configuration. Each detector element 20 produces an electrical signal that represents the intensity of an impinging x-ray beam. As the x-ray beam passes through a patient 22, the beam is attenuated. During a scan to acquire x-ray projection data, gantry 12 and the components mounted thereon rotate about a z-axis center of rotation 24.

Rotation of gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high speed image reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

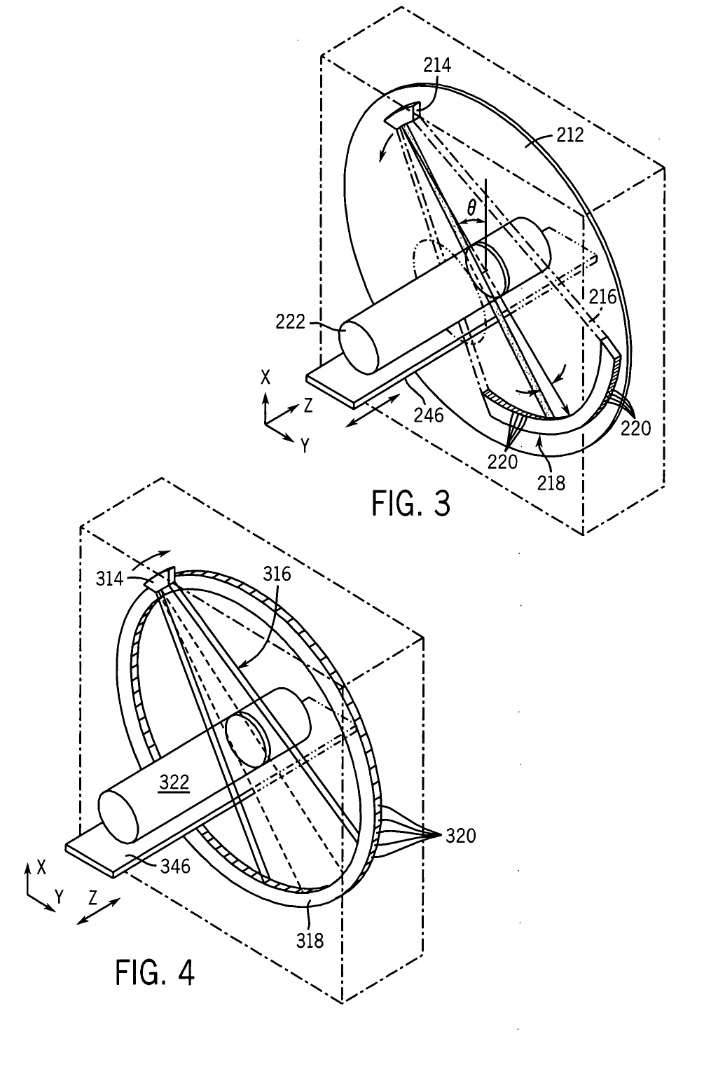

FIG. 3 illustrates a source-detector assembly 210 which is a specific embodiment of the source-detector assembly 110 shown schematically in FIG. 2. Assembly 210 illustrates the particular case of a so-called third generation, fan beam CT system. In the assembly 210, a gantry assembly 212 corresponds to the gantry 112 of FIG. 1. An x-ray source 214 generates a fan beam 216 of x-rays directed toward a detector array 218, which is also affixed to the gantry assembly 212. Array 218 comprises individual detector elements 220 that detect x-rays emitted by source 214. The subject 222, table 246, and subject aperture 248 correspond to subject 122, table 146, and aperture 148 as described with respect to FIG. 2. In operation, assembly 212 rotates around the axis Z passing through subject 222 and perpendicular to the plane XY. Source 214 can thereby be transported completely around subject 222 along a circular path. Detector array 218, being fixed with respect to source 214, is also transported around subject 222 and thus remains opposite source 214.

Rotation of the gantry assembly 212 around the subject 222 results in x-ray data being acquired by detector elements 220 for a range of view angles θ. A typical detector array 218 may comprise several hundred individual detector elements 220, such as 888 individual elements 220. The array 218 is positioned on the gantry 212 at a distance of, for example, 0.949 meter (m) from the x-ray source 214. The circular path of source 214 has a radius of, for example, 0.541 m. Particular values of these parameters are not critical to the present invention and may be varied according to well-known principles of CT system design.

One complete gantry rotation for the gantry 212 may comprise, for example, 984 view angles. Source 214 is thereby positioned to illuminate the subject 222 successively from 984 different directions θ and the detector array 218 generates x-ray data at each view angle θ, from which projection data for 984 separate projection views are acquired.

FIG. 4 illustrates a source-detector assembly 310 for a so-called fourth generation fan beam CT system. An x-ray source 314, like source 214 and as a further example of source 114, generates a fan beam 316 of x-rays directed toward a detector array 318. The array 318 comprises detector elements 320 that generate x-ray attenuation data indicating internal structural information about a subject 322. The fourth generation case of FIG. 4 differs from the third generation case, in that the detector array 318 extends completely around the z-axis and does not rotate. The x-ray source 314 does rotate around the z-axis and traverses a circular path around the subject 322. The detector array 318 may translate axially (in the Z direction) to provide x-ray data for a particular axial slice. Alternatively, the array 318 may be fixed axially as well as rotationally, and positioning of the subject 322 may be achieved by axial translation of a table 346.

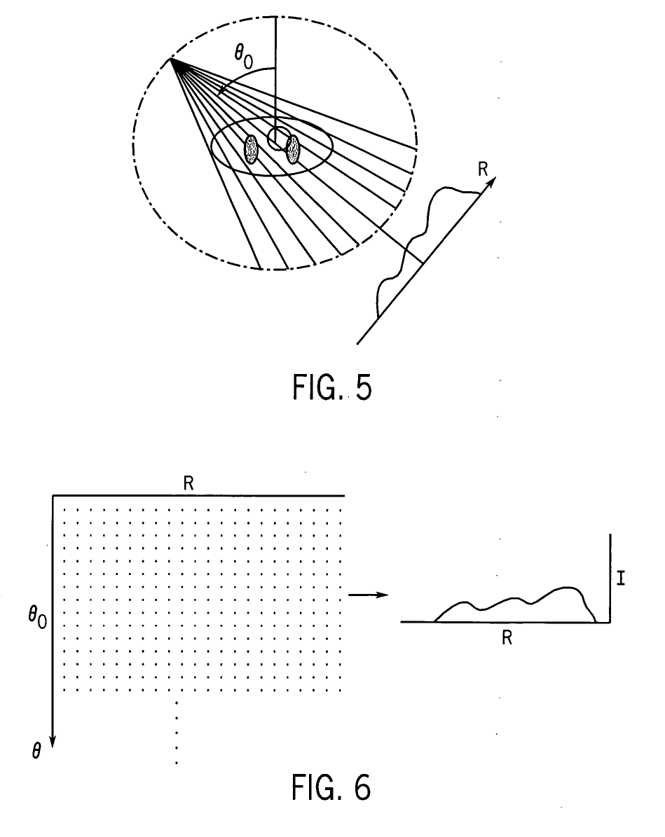

In the fan beam case, the data acquisition cycle is sometimes called an axial scan. The projection data for an axial scan is comprised of a set of projection views all acquired at the same axial position z₀. As shown in FIG. 5, each projection view is acquired at a specific view angle θ and each detector attenuation measurement is at a location R in the detector array. As shown in FIG. 6, data from an axial scan may be stored in a two dimensional array called a “sinogram.” One dimension of the sinogram corresponds to angular position of the fan beam, or view angle θ. The other dimension corresponds to positions of the detector elements (R) of the detector array. The detector array in a fan beam CT system (array 218 of FIG. 3 or array 318 of FIG. 4) generally comprises a single row of detector elements. Therefore, each row of the sinogram corresponds to a discrete view angle θ and a single axial position z₀.

A sinogram obtained from an axial scan is a collection of projection views of the subject at the position z₀. Here the term “projection view” means such a row of projection data corresponding to a given view angle θ and representing the imaged subject at a single axial position z₀. Well known tomographic image reconstruction procedures utilize as their principal inputs a complete set of such projection views (discretized in θ, but all consisting of data values for the same axial position z₀). The projection views are processed by such tomographic techniques to reconstruct a slice image depicting the internal features of the subject in a slice located at the position z₀.

FIG. 5 illustrates the correspondence between a particular view angle θ₀ for the x-ray source and the generation of a well defined row R of projection data. In the fan beam case, as noted above, the detector data from the detector array may convert directly into a single row of projection data for a projection view at view angle θ₀. This correspondence results because the detector array provides a single row of detector data representing intensities (I) of the x-rays impinging upon the detector elements. These intensity values (I) indicate attenuation information for the subject at the axial position z₀.

FIG. 6 shows how the projection data for the particular view angle θ₀ is stored in a corresponding row of the sinogram. Each row of this sinogram thus constitutes a projection view that indicates attenuation information (I) for a distinct view angle θ, at the same axial position z₀. Once the sinogram is filled with projection views for all the discrete view angles θ around the subject, then a suitable CT tomographic image reconstruction algorithm is applied to reconstruct a cross-sectional image of the subject.

Tomographic image data for a three-dimensional representation may comprise image data for several slice images at a succession of axial positions (so-called “stacked 2-D slices” or “stacked slice images”). One way to obtain these multiple slice images is to acquire corresponding sinogram data sets slice by slice using, for example, a fan beam CT imaging system (such as the system of FIG. 3 or the system of FIG. 4). However, the preferred method is to use what is called a helical scan.

Helical scanning relaxes the requirement of axial scanning systems that fix the axial position of the gantry at a single z-axis point throughout the data collection cycle. Instead, the entire gantry (source and detector array) translates axially (in the z direction) relative to the patient while the gantry is being rotated. A single scanning operation (i.e., continuous rotation of the gantry) can thereby cover the entire organ or structure under study. The projection views thus acquired may be processed to form a plurality of sinogram data sets at discrete slice locations along the z-axis. Such processing is well known in the art as exemplified by the method disclosed in U.S. Pat. No. 5,270,923 issued on Dec. 14, 1993 and entitled “Computed Tomographic Image Reconstruction Method For Helical Scanning Using Interpolation Of Partial Scans For Image Construction” which is incorporated herein by reference.

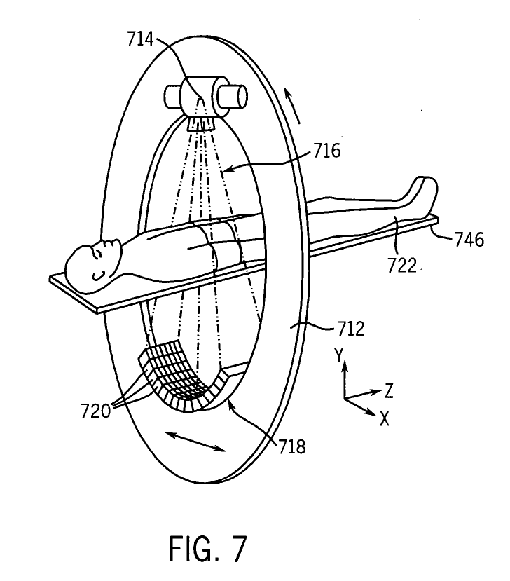

FIG. 7 illustrates a source-detector assembly 710 for a desirable alternative to helical fan beam scanning, called helical cone beam scanning. The principal features of assembly 710 are analogous to the components of assemblies 210 and 310 in FIGS. 2 and 3, respectively. A gantry 712 supports an x-ray source 714 that generates an imaging x-ray beam 716. However, unlike the fan beams 216 and 316 described previously, the beam 716 is a so-called cone beam that spreads (or “fans”) in two generally orthogonal directions as the beam is projected away from the source 714.

The assembly 710 of FIG. 7 corresponds to the third generation axial assembly 210 of FIG. 2 in which both the source 714 and the detector array 718 are transported around a subject 722 along respective circular paths as the gantry 712 rotates. Unlike detector array 218, however, the detector array 718 is a so-called multi-row, or two-dimensional detector comprising several rows of detector elements 720. Each row of the array 718 extends circumferentially with respect to the gantry rotation, and the succession of rows extends axially with respect to the gantry z-axis of rotation. The array 718 thereby provides a two dimensional detection area, which corresponds to the spread of the cone beam 716 in two complementary directions. A helical/cone beam CT scanning system provides advantages over the fan beam for acquiring a 3D image. The multi-row detector such as detector array 718 can collect several times more x-ray data during each gantry rotation.

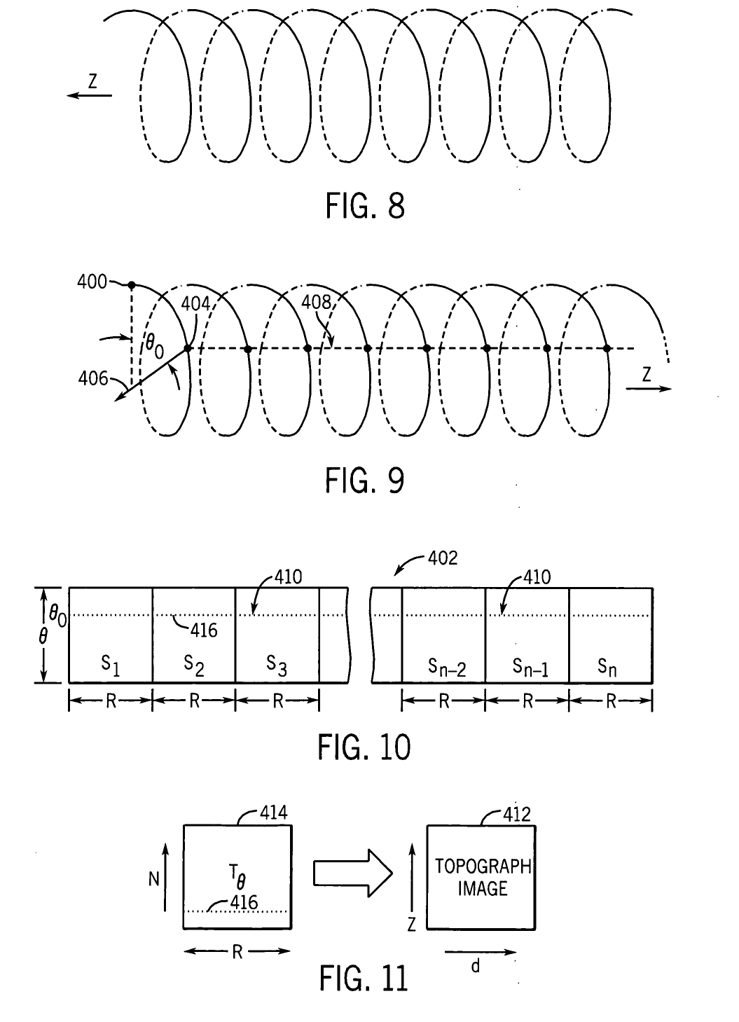

In both fan beam and cone beam helical scanning the x-ray source follows a helical path given by
θ(0)=ωt
Z(0)=(pΔ_{d}/2π)ωt
where θ(0) is the view angle, Z(0) is the axial position of the source, ω is the rate of gantry rotation, and p (for “pitch”) is the axial translation per gantry rotation, as a fraction of detector spacing Δ_{d}. The helical path of the x-ray source during a helical scan is illustrated in FIG. 8.

One aspect of the present invention is a new method of using the sinogram data sets produced during a helical scan to reconstruct a high resolution digital subtraction angiogram. This concept will be explained first with sinogram data produced during a helical scan using either the 3^{rd} or 4^{th} generation fan beam systems of FIG. 3 or 4.

Referring particularly to FIGS. 9 and 10 when a helical fan beam scan is performed the x-ray source starts at a starting point (Z=0 and θ=0) as indicated at point 400 and follows a helical path relative to the subject of the examination. At many points along this helical path projection views are acquired as discussed above at a succession of view angles (θ=0 to 360°). These are stored in a sinogram indicated at 402. If the source revolves around the subject n times during the helical scan, n sinogram data sets (S) are acquired and stored. Each sinogram data set S will contain projection views from the same set of view angles (θ=0° through 360°). For example, a projection view acquired at view angle θ₀ during the first gantry revolution as indicated by point 404 will “see” the subject from the vantage point indicated by arrow 406. During subsequent gantry revolutions projection views from the same view angle θ₀ will be acquired as indicated by the points connected by dotted line 408 in FIG. 9. These same projection views are stored on the same line of each of the sinogram datasets S₁ through Sₙ, as indicated by the dotted line 410 in FIG. 10. The difference between these projection views at θ₀ is the z-axis location of the x-ray source when they were acquired. This can be expressed as follows:
Z_{N}=Z₁+pΔ_{d}N, (1)
where Z₁ is the location of the first projection view, p is the pitch of the helical scan, Δ_{d} is detector spacing, and Z_{N} is the z-axis location of the same projection in the N^{th} gantry revolution.

Referring particularly to FIGS. 10 and 11, a topograph image 412 is produced by first forming a topographic plane data set T_{θ} indicated at 414. This is done by selecting from each sinogram data set S₁ through Sₙ the projection view acquired at the same projection angle θ. In the example discussed above, the views at θ₀ are selected and the topograph will view the subject from this angle. For example, the θ₀ projection view from sinogram data set S₂ is indicated by dotted line 416.

The topograph image 412 is then produced by mapping the individual attenuation measurements in each projection view of topographic data set T_{θ} to a specific pixel location. Each attenuation measurement has coordinates N and R and these are converted to positions along respective axes z and d in the topograph 412. The z-axis location is given by equation (1) above and the d axis location is determined in the usual fashion by the geometry of the detector array (e.g., Δ_{d}) and the gantry. The attenuation measurements control the intensity of their corresponding pixels in the topograph 412. The axis d is in the x,y plane, perpendicular to the z-axis and it is perpendicular to the selected view angle θ₀.

When the topograph image 412 is displayed one sees a 2D projection image of the subject from the selected view angle θ₀. The view angle θ₀ can be selected to produce a topograph image 412 that lies in either the xz plane or the yz plane, or many angles therebetween. Topograph images 412 can be reconstructed at many different view angles θ using the same acquired data sets, and these can be sequentially displayed to rotate the subject.

In the embodiment described above a single spiral data acquisition pattern is produced. This pattern is applicable to a fan beam system in which a single row of detector elements acquire data at a single z-axis location during each view acquisition. It can be appreciated that when a cone beam system is employed, each view acquisition acquires data at a plurality of z-axis locations corresponding to the plurality of rows in the 2D detector array 718. As a result a plurality of interleaved spiral patterns of data are acquired and stored in a corresponding plurality of sets of sinograms. Thus, instead of the single set of sinograms 402 illustrated in FIG. 10, a plurality of such sets are acquired during a cone beam helical scan. However, the z-axis location of corresponding data points in each data set differs by the z-axis spacing between rows of detector elements. Stated another way, the starting location Z₁ in equation (1) is different for the set of sinograms produced by each row of detector elements. The topographic plane data set T_{θ} is formed by selecting from each sinogram data set the projection view acquired at the selected projection angle θ, but the starting location Z₁ in equation (1) used to map each attenuation value to a pixel location in the topograph image 412 will depend on which detector row its measurement was made.

In the embodiments described above only views stored at a selected view angle θ₀ are employed to produce the topograph image 412. However, the resolution of the topograph 412 can be doubled in the z-axis direction by also employing the views acquired on the opposite side of the gantry (i.e., θ₀+180°). That is, the attenuation data acquired at view angle θ₀+180° sees the subject at the same view angle θ₀ , but from the opposite side of the subject and at z-axis locations interleaved with the attenuation data acquired at view angle θ₀. Because the attenuation data is acquired with a fan beam having non-parallel rays, however, it is necessary to rebin the raw “diverging rays” projection data to form parallel rays. Such a rebinning step is described, for example, in U.S. Pat. No. 5,216,601 issued on Jun. 1, 1993 and entitled “Method For Fan Beam Helical Scanning Using Rebinning” which is incorporated herein by reference.

In the above-described embodiments the acquired views stored in the sinogram arrays 402 are used to reconstruct the topograph image 412. In commercially available CT systems the data in these sinograms is processed first to form complete sinograms at specific z-axis slice locations. This is an interpolation process as described in the above cited U.S. Pat. No. 5,270,923, and the result is a set of sinogram data sets at specific z-axis slice locations. These slice sinogram data sets may be used in the same manner as described above to form the topographic plane data set 414. However, equation (1) is not used to map attenuation values to pixel locations in the topograph image 412. Instead, all the attenuation values in a row of the topographic plane data set 414 are mapped to a z-axis location corresponding to its slice location. In this case the resolution of the topograph 412 is determined by the z-axis spacing of the slice sinogram data sets.

Another aspect of the present invention is the reconstruction of images from acquired sinogram data sets in which artifacts caused by metallic objects in the field of view are substantially suppressed. This is achieved by acquiring a first set of sinogram data sets as described above and then acquiring a substantially identical set of sinogram data sets after the IV injection of a contrast agent. Artifact suppression is achieved by subtracting the acquired projection views in the first set of sinogram data sets from the corresponding projection views in the second set of sinogram data sets to produce a set of difference sinogram data sets. These difference sinogram data sets are then employed to produce the topograph image 412 as described above.

It is also a discovery of the present invention that image artifacts may be substantially suppressed in tomographically reconstructed images by employing the difference sinogram data sets. Rather than reconstructing tomographic images from the respective first and second sets of sinogram data sets and then subtracting the resulting two images as is done with computed tomography angiography (CTA), according to the present invention the corresponding acquired projection views are subtracted prior to image reconstruction. An important requirement for this to work properly is that corresponding projection views in the two sets of sinogram data sets are acquired at substantially the same projection angle (θ) and z-axis location. To accomplish this the starting point (θ, z) of the two helical scans should be substantially identical and the helical scan paths should be substantially the same.

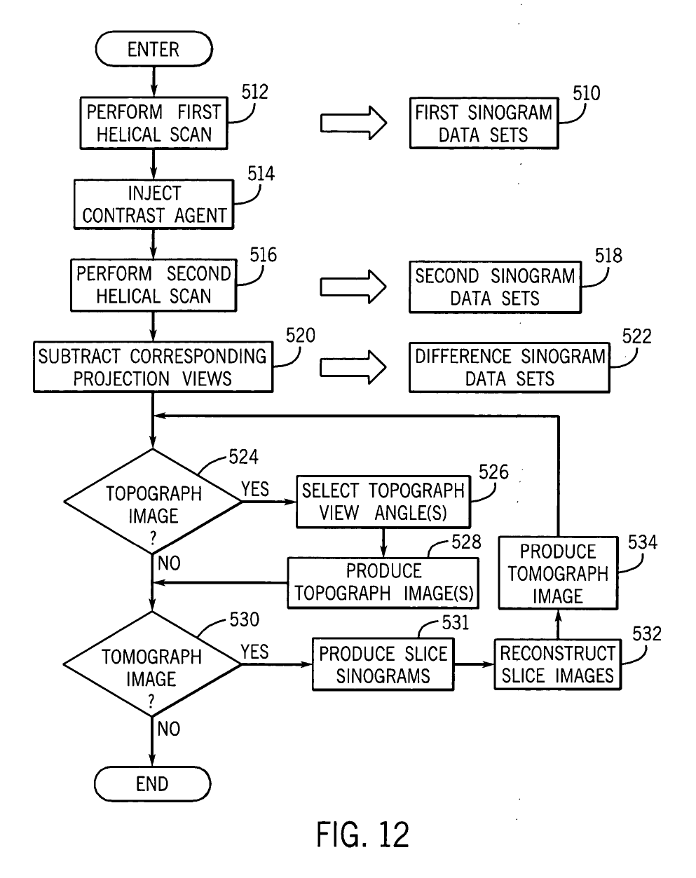

Referring particularly to FIG. 12, a programmed procedure for implementing a preferred embodiment of the invention on the CT imaging system of FIGS. 1 and 2 is illustrated. A first step is to acquire a first set of sinogram data sets 510 by performing a first helical scan 512 as described above. The number and size of these sinogram data sets will depend on factors such as the number of revolutions of the gantry during the scan, the pitch of the helical scan, the number of detector elements in a row and the number of rows of detector elements in the CT system.

The patient is then injected with a suitable contrast agent as indicated at process block 514. After a short period of time during which the contrast agent flows into the field of view and alters the x-ray attenuation characteristics of the tissues of interest, a second helical scan is performed as indicated at process block 516 to produce a second set of sinogram data sets 518. As mentioned above, it is important that these two helical scans are the same and are geometrically registered with each other so that corresponding projection views in the two data sets 510 and 518 are acquired at the same projection angles θ and z-axis locations.

As indicated at process block 520, the next step is to subtract corresponding projection views in the two sets of sinogram data sets 510 and 518. This results in difference sinogram data sets 522 which depict the difference in x-ray attenuation of the subject tissues before and after contrast injection.

The difference sinogram data sets 522 can be processed in a number of different ways to produce a variety of images. If a topographic image is to be produced as indicated at decision block 524, the operator is prompted to select a topographic view angle at process block 526. In the alternative a number of view angles may be selected or a range of view angles may be selected. The topographic image, or images are then produced as described above by selecting from the difference sinogram data sets 522 the projection views at the selected view angle (or view angles) as indicated at process block 528. The topographic images may be displayed so that the operator may see a radiograph-like projection of the subject from the selected view angle or angles. These topographic images may also be stored for later viewing.

If a tomographic image is to be produced as indicated at decision block 530, the difference sinogram data sets are interpolated to produce discrete slice sinogram data sets at specific slice intervals along the z-axis as indicated at process block 531. This is a well known procedure in the art as discussed above for converting data acquired with a helical scan to sinograms at successive slice locations along the z-axis. As indicated at process block 532, a conventional tomographic image reconstruction is then performed with each slice sinogram data set. A well known filtered backprojection method is employed in the preferred embodiment. The reconstructed slice images may be displayed separately as indicated at process block 534, but preferably a three-dimensional image is produced by concatenating the 2D slice images. The 3D image can be displayed by projecting it at any view angle onto the viewing plane, or slices through the 3D image at any location and angle may be viewed.

The present invention provides a number of valuable tools for the physician. In a single study it provides a 2D or 3D computed tomography image which is known for its high definition anatomical depiction of the subject. In addition, high resolution 2D topograph images that exceed the resolution of the current gold standard DSA images may be produced. The difference sinogram data sets can also be reconstructed into 3D tomographic images that are free of bone and metal artifacts and can be manipulated for viewing in any plane. Furthermore, the IV injection of contrast agent according to the present invention avoids the need for direct arterial catheterization required by DSA and, therefore, does not carry with it the attendant medical risks and the high costs of qualified medical personnel needed by the catheterization procedure.

1. A method for producing an image of a subject with an x-ray system, the steps comprising:
a) acquiring a data set comprised of a set of projection views of the subject acquired at a corresponding set of view angles;
b) injecting a contrast agent into the subject to modify the x-ray attenuation of tissues in the subject;
c) repeating step a) to acquire a second data set comprised of projection views of the subject acquired at the same view angles;
d) subtracting corresponding projection views in the two data sets to form a difference data set; and
e) reconstructing an image of the subject from the difference data set.

2. The method as recited in claim 1 in which each projection view is a one-dimensional set of attenuation values produced by a fan beam of x-rays and the reconstructed image is a two-dimensional tomographic image.

3. The method as recited in claim 1 in which each projection view is a two-dimensional set of attenuation values produced by a cone beam of x-rays and the reconstructed image is a three-dimensional tomographic image.

4. The method as recited in claim 1 in which step a) includes revolving an x-ray source a plurality of times around the subject and providing relative axial motion between the x-ray source and the subject; and
in which the projection views acquired in step c) are at both the same view angles and same relative axial positions as the projection views in the first data set.

5. The method as recited in claim 4 in which step e) includes:
i) forming a topographic plane data set by selecting from said difference data set projection views acquired at substantially the same view angle (0) and at successive relative axial positions; and
ii) mapping the values in the topographic plane data set to pixel locations in a topograph image.

6. The method as recited in claim 5 in which step i) also includes selecting from said difference data set projection views acquired at substantially the opposite view angle (θ±180°).

7. The method as recited in claim 5 in which step a) includes providing the relative axial motion as the x-ray source revolves around the subject to perform a helical scan.

8. The method as recited in claim 7 in which step a) includes interpolating acquired projection views such that the projection views in the data set are at discrete axial slice locations; and
in which the projection views acquired in step c) are at both the same view angles and same discrete axial slice locations as the projection views in the first data set.

9. A method for producing a topograph image of a subject with an x-ray system, the steps comprising:
a) performing a helical scan of the subject to acquire a plurality of successive sinogram data sets, each sinogram data set storing a plurality of projection views acquired at a corresponding plurality of view angles;
b) forming a topographic data set by selecting from successive sinogram data sets projection views acquired at substantially the same view angle (θ); and
c) mapping values in the topographic data set to pixel locations in the topographic image.

10. The method as recited in claim 9 in which step b) includes selecting from successive sinogram data sets projection views acquired at substantially the opposite view angle (θ±180°).

11. The method as recited in claim 10 in which the helical scan is performed with a fan beam x-ray source and step b) further includes rebinning diverging rays in the fan beam projection views to form parallel ray projection views.

12. The method as recited in claim 10 in which the helical scan is performed with a cone beam x-ray source and step b) further includes rebinning diverging rays in the cone beam projection views to form parallel ray projection views.

13. The method as recited in claim 9 in which step c) includes setting the brightness of pixels in the topographic image in accordance with the corresponding values mapped from the topographic data set.

14. The method as recited in claim 9 in which the successive sinogram data sets are acquired at successive axial locations and one dimension of the topographic image is indicative of axial location.

15. The method as recited in claim 14 in which the data in each sinogram data set is indicative of data acquired at substantially the same axial location.

16. The method as recited in claim 14 in which the data in each projection view in each of said sinogram data sets is acquired at substantially the same axial location, but the axial location of data in each projection view is acquired at a different axial location.
